(19) United States
(12) Patent Application Publication
(10) Pub. No.: US 2008/0182908 A1
(43) Pub. Date: Jul. 31, 2008

(54) PHARMACEUTICAL COMPOSITIONS COMPRISING MEMANTINE

(76) Inventors: Vinita Umashankar Vyas, Cuttack (IN); Ravi Kumar Namballa, Hyderabad (IN); Raghupathi Kandarapu, Warangal (IN); Srinivas Irukulla, Hyderabad (IN); Indu Bhushan, Hyderabad (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 12/018,974

(22) Filed: Jan. 24, 2008

Related U.S. Application Data
(60) Provisional application No. 60/914,871, filed on Apr. 30, 2007.

(30) Foreign Application Priority Data
Jan. 25, 2007 (IN) 164/CHE/2007

(51) Int. Cl. A61K 31/136

(52) U.S. Cl. 514/661

(57) ABSTRACT

Pharmaceutical compositions comprising memantine or a salt thereof, from which less than about 80% of the contained memantine dissolves into a pH 1.2 aqueous medium within about 60 minutes.

### INTRODUCTION TO THE INVENTION

The present invention relates to immediate release solid oral dosage forms comprising memantine or pharmaceutically acceptable salts thereof, wherein said dosage form has a release rate of less than about 80% of active ingredient, within about the first 60 minutes, in an environment that dosage form is likely to encounter when administered to humans.

Memantine is a systemically active noncompetitive NMDA (N-methyl-D-aspartate) receptor antagonist having a low to moderate affinity for the receptor and rapid blocking/unblocking kinetics. A chemical name for memantine is 1-amino-3,5-dimethyladamantane. A structural formula for memantine is (1).

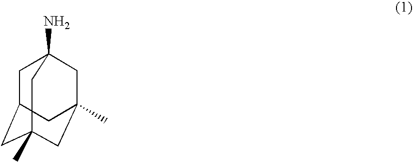

The salt memantine hydrochloride occurs as a fine white to off-white powder and is highly soluble in water. The molecular formula is C₁₂H₂₁N.HCl and the molecular weight is 215.76.

Memantine hydrochloride is commercially available in the form of NAMENDA™ oral tablets and NAMENDA™ oral solution. NAMENDA™ oral tablets are available as capsule-shaped, film coated tablets containing 5 mg or 10 mg of memantine hydrochloride. The tablets also contain the inactive ingredients microcrystalline cellulose, lactose monohydrate, colloidal silicon dioxide, talc and magnesium stearate. In addition the following inactive ingredients are also present as components of the film coating: hypromellose, triacetin, titanium dioxide, FD&C yellow #6 and FD&C blue #2 (5 mg tablets), and iron oxide black (10 mg tablets).

NAMENDA oral solution contains memantine hydrochloride in a strength equivalent to 2 mg of memantine hydrochloride in each milliliter.

Memantine and salts thereof are indicated for treatment of central nervous systems diseases such as Alzheimer's disease.

An immediate release formulation, include improved ease of administration and increased flexibility in drug administration.

Memantine is disclosed in U.S. Pat. Nos. 5,061,703 and 5,382,601, and U.S. Patent Application Publication Nos. 2004/0254251, 2006/0198884, 2006/0142398, 2006/0051416, 2006/0002999 and 2007/0065512 disclose compositions of memantine. U.S. Pat. No. 5,614,560 and U.S. Patent Application Publication No. 2006/0020042 disclose methods of treatment using memantine.

The recommended starting dose of memantine hydrochloride is 5 mg once daily. The recommended target dose is 20 mg/day. The dose should be increased in 5 mg increments to 10 mg/day (5 mg twice a day), 15 mg/day (5 mg and 10 mg as separate doses), and 20 mg/day (10 mg twice a day), and the minimum recommended interval between dose increases is one week.

The existence of immediate release formulations of different strengths of memantine would therefore, allow ease and convenience in dosing during both up-titration phase and during maintenance at the higher therapeutic dose levels.

Sometimes a dosing regimen of memantine of twice a day using immediate release tablets may be undesirable because patient compliance decreases as the frequency of taking drug increases. Moreover administration of an immediate release tablet sometimes may lead to a greater frequency of adverse events due to a faster rate of absorption. Hence it is advantageous to reduce the rate of absorption by controlling the drug release to some extent. As drug release becomes slower, the dose dumping and reduction in side effects will be reduced.

### SUMMARY OF THE INVENTION

The present invention relates to immediate release solid oral dosage forms comprising memantine or pharmaceutically acceptable salts thereof, wherein said dosage forms release less than about 80% of contained memantine, within about the first 60 minutes, in an environment that dosage form is likely to encounter when administered to humans.

In an embodiment the present invention includes processes of preparing compositions having an immediate release profile.

In an embodiment the present invention includes methods of using the compositions of the present invention.

An embodiment of the invention provides a pharmaceutical composition comprising memantine or a salt thereof, wherein less than about 80 percent of contained memantine dissolves within about 60 minutes after immersion in a pH 1.2 aqueous medium.

Another embodiment of the invention provides a pharmaceutical composition comprising memantine hydrochloride and a polymer, wherein less than about 80 percent of contained memantine dissolves within about 60 minutes after immersion in a pH 1.2 aqueous medium.

A further embodiment of the invention provides a pharmaceutical composition comprising memantine hydrochloride and a cellulose derivative polymer, wherein less than about 80 percent of contained memantine dissolves within about 60 minutes after immersion in a pH 1.2 aqueous medium, the composition producing memantine Cₘₐₓ values about 10 ng/mL to about 20 ng/mL, AUC₀₋ₜ values about 720 ng·hour/mL to about 1150 ng·hour/mL, and AUC_{0-α} values about 850 ng·hour/mL to about 1330 ng·hour/mL, after oral administration of a single 10 mg memantine hydrochloride dose to healthy humans.

### DETAILED DESCRIPTION OF THE INVENTION

The present invention relates to immediate release solid oral dosage forms comprising memantine or pharmaceutically acceptable salts thereof, wherein said dosage forms release less than about 80% of contained memantine, within about the first 60 minutes, in an environment that dosage form is likely to encounter when administered to humans. The formulations may be administered for once daily or twice daily dosing.

In an embodiment the present invention includes processes of preparing compositions comprising memantine.

The pharmaceutical compositions may be prepared using various formulation techniques such as physical mixing, blending, wet granulation, dry granulation, direct compression, fluid bed granulation, etc., and any combinations thereof

In an embodiment, the present invention comprises immediate release solid oral dosage forms comprising memantine or pharmaceutically acceptable salts thereof, wherein said dosage forms release less than about 80% of contained memantine, within about the first 60 minutes when tested in vitro in 900 ml of pH 1.2 dissolution medium using test method 711 “Dissolution” in United States Pharmacopeia 29, United States Pharmacopeial Convention, Inc., Rockville, Md., 2005 (“USP”), and type I apparatus. In various embodiments, less than about 70%, or less than about 60%, or less than about 50%, of the contained memantine is released within about 60 minutes in the test.

An environment that a dosage form is likely to encounter when administered to human comprises but is not limited to simulated gastric fluid (SGF) with or without pepsin, simulated intestinal fluid (SIF) with or without pancreatin, 0.01 N HCl, pH 1.2, 4.5, 5.5, 6.0, 6.8, 7.2, and 7.4 buffers, pH 2.1 SGF, pH 5.0 and 4.5 acetate buffers, pH 4.5 ammonium acetate buffer, pH 5.0 Fed State Simulated Intestinal Fluid (FeSSIF), pH 6.5 Fasted State Simulated Intestinal Fluid (FA SSIF), pH 1.5 HCl buffer, etc.

In an embodiment, the present invention includes methods of using the compositions of the present invention.

According to the present invention, memantine may be used in the form of a free base or a pharmaceutically acceptable salt. The pharmaceutically acceptable salts of memantine include but are not limited to acid addition salts, such as those made with hydrochloric, methylsulfonic, hydrobromic, hydroiodidic, perchloric, sulfuric, nitric, phosphoric, acetic, propionic, glycolic, lactic, pyruvic, malonic, succinic, maleic, fumaric, tartaric, citric, benzoic, carbonic cinnamic, mandelic, methanesulfonic, ethanesulfonic, hydroxyethanesulfonic, benzenesulfonic, p-toluene sulfonic, cyclohexanesulfamic, salicylic, p-aminosalicylic, 2-phenoxybenzoic, and 2-acetoxybenzoic acids, and salts made with saccharin.

The present invention further includes use of any individual enantiomers, diastereomers, racemates, polymorphs, solvates, such as hydrates and those formed with organic solvents, and other isomers.

In an embodiment the invention includes memantine in the form of its hydrochloride salt, i.e., memantine hydrochloride, in amounts ranging from about 2.5 mg to about 100 mg per unit dosage form.

The different physicochemical properties of the active ingredient as well as excipients are to be considered, as these properties affect processing and the formulation properties of the compositions. Various physicochemical properties include, but are not limited to, particle size of the active ingredient, density (including bulk density and tapped density), compressibility index, Hausner's ratio, angle of repose, etc.

These physicochemical properties not only affect processes of the preparing the pharmaceutical compositions but also can affect the performance of the pharmaceutical product both in vitro and in vivo.

The percentages of particles with different sizes that exist in a total powder, is the particle size distribution. It is represented in certain ways. Particle size is the maximun dimension of a particle, frequently expressed in micrometers. Particle size distributions can be expressed in terms of D₁₀, D₅₀, D₉₀ and D[4,3]. The D₁₀, D₅₀ and D₉₀ represent the 10th, median or the 50th percentile, and the 90th percentile of the particle size distribution, respectively, as measured by volume. That is, the D₁₀, D₅₀, D₉₀ are values of the distribution such that 10%, 50%, and 90% of the particles have a size of this value or less or are the percentages of particles smaller than that size. D₅₀ also known as median diameter of particles. It is one of the important parameters representing characteristics of particle of powder. For a sample, if D₅₀=5 μm, it means that 50% of the particles are smaller than 5 μm. Similarly, if D₁₀=5 μm, 10% of the particles are less than or equal to 5 μm and if D₉₀=5 μm, 90% of the particles have sizes less than or equal to 5 μm. D[4,3] is the volume moment mean of the particles or the volume weighted particle size.

In an embodiment the invention includes a defined particle size distribution for memantine hydrochloride. The particle size distribution includes a plurality of memantine hydrochloride particles where D₅₀ (mean particle size) is about 25 μm to about 100 μm, D₁₀ is about 1 μm to about 25 μm, and D₉₀ is about 150 μm to about 300 μm.

In an embodiment, particles of memantine hydrochloride have a ratio of D₁₀ to D₉₀ ranging from about 1:1 to about 1:30. In another embodiment, particles of memantine hydrochloride have a ratio of D₁₀ to D₉₀ ranging from about 1:15 to about 1:25. In a further embodiment, a ratio of D₁₀ to D₉₀ is about 20.

In an embodiment, the invention includes bulk densities of the active ingredient ranging from about 0.25 g/ml to about 0.45 g/ml and tapped densities of the active ingredient ranging from about 0.35 g/ml to about 0.55 g/ml.

In an embodiment the invention includes compositions to achieve desired release rates, which may be formulated using polymeric coating or matrixes.

An immediate release dosage form optionally has a coating applied over the surface of a core particle.

In an embodiment, examples of coating materials used for immediate release compositions include but are not limited to hydrophilic polymers such as hydroxypropyl methylcellulose, methacrylates, povidone, hydroxyethyl cellulose, copovidones such as Plasdone™ S-630, and gums such as xanthan gum, acacia, guar gum, etc.

Depending upon the hydrophilic (erodable or nonerodable) or hydrophobic nature of the matrix, the matrix may comprise materials that swell upon contact with gastric fluid to a size that is large enough to promote retention in the gastric fluid.

Suitable polymers include but are not limited to cellulose polymers and derivatives, including, but not limited to, hydroxymethyl cellulose, hydroxyethyl cellulose, hydroxypropyl methylcellulose, carboxymethyl cellulose, and microcrystalline cellulose polysaccharides and their derivates, cellulose esters, carboxyvinyl esters, or acrylic or methacrylic esters, polyalkylene oxides, polyethylene glycols, chitosan, polyvinyl alcohol, xanthan gum, maleic anhydride copolymers, polyvinylpyrrolidone, starch and starch based polymers, maltodextrins, poly(2-ethyl-2-oxazoline), poly(ethyleneimine), polyurethane hydrogels, and crosslinked polyacrylic acids and their derivatives.

In an embodiment the in vitro dissolution release rate of the drug depends on the composition of matrix or coating compositions.

In an embodiment, compositions having an immediate release rate comprise coating materials ranging from about 3% w/w to about 25% w/w of the total weight of the compositions.

In another embodiment, compositions having an immediate release rate comprise polymer concentrations ranging from about 5% w/w to about 55% w/w of the total weight of the compositions.

In an embodiment, the invention includes compositions, which are formulated into solid oral dosage forms such as tablets, capsules, granules, etc.

In another embodiment, the invention includes compositions, which are formulated into liquid oral dosage forms.

The compositions of the present invention further comprise one or any combinations of pharmaceutical excipients such as, but not limited to, diluents, binders, disintegrants, glidants, lubricants, colorants, flavouring agents, solvents, film forming polymers, plasticizers, opacifiers, antiadhesives, and polishing agents.

### Diluents:

Various useful diluents include but are not limited to starches, lactose, mannitol, pearlitol SD 200, cellulose derivatives, confectioners sugar and the like. Different grades of lactose include but are not limited to lactose monohydrate, lactose DT (direct tableting), lactose anhydrous, Flowlac™ (available from Meggle Products), Pharmatose™ (available from DMV) and others. Different grades of starches include but are not limited to maize starch, potato starch, rice starch, wheat starch, pregelatinized starch (commercially available as PCS PC10 from Signet Chemical Corporation) and Starch 1500, Starch 1500 LM grade (low moisture content grade) from Colorcon, fully pregelatinized starch (commercially available as National 78-1551 from Essex Grain Products) and others. Different cellulose compounds that can be used include crystalline cellulose and powdered cellulose. Examples of crystalline cellulose products include but are not limited to CEOLUS™ KG801, Avicel™ PH 101, PH102, PH301, PH302 and PH-F20, microcrystalline cellulose 114, and microcrystalline cellulose 112. Other useful diluents include but are not limited to carmellose, sugar alcohols such as mannitol, sorbitol and xylitol, calcium carbonate, magnesium carbonate, dibasic calcium phosphate, and tribasic calcium phosphate.

In an embodiment the invention includes a concentration range of diluents ranging from about 5% w/w to about 85% w/w of the total composition.

### Binders:

Various useful binders include but are not limited to hydroxypropyl cellulose (Klucel™ LF), hydroxypropyl methylcellulose (HPMC or hypromellose, e.g., Methocel™), polyvinylpyrrolidone or povidone (PVP-K25, PVP-K29, PVP-K30, PVP-K90), copovidones such as Plasdone™ S 630, powdered acacia, gelatin, guar gum, carbomer (e.g. Carbopol), methylcellulose, polymethacrylates, and starch.

### Disintegrants:

Various useful disintegrants include but are not limited to carmellose calcium (Gotoku Yakuhin Co., Ltd.), carboxymethylstarch sodium (Matsutani Kagaku Co., Ltd., Kimura Sangyo Co., Ltd., etc.), croscarmellose sodium (FMC-Asahi Chemical Industry Co., Ltd.), crospovidone, examples of commercially available crospovidone products including but not limited to crosslinked povidone, Kollidon™ CL [manufactured by BASF (Germany)], Polyplasdone™ XL, XI-10, and INF-10[manufactured by ISP Inc. (USA)], and low-substituted hydroxypropylcellulose. Examples of low-substituted hydroxypropylcellulose include but are not limited to low-substituted hydroxypropylcellulose LH11, LH21, LH31, LH22, LH32, LH20, LH30, LH32 and LH33 (all manufactured by Shin-Etsu Chemical Co., Ltd.). Other useful disintegrants include sodium starch glycolate, colloidal silicon dioxide, and starch.

### Glidants:

Various useful glidants or antisticking agents include but are not limited to talc, silica derivatives, colloidal silicon dioxide and the like and mixtures thereof.

### Lubricants:

Various lubricants that can be used include but are not limited to stearic acid and stearic acid derivatives such as magnesium stearate, calcium stearate, zinc stearate, sucrose esters of fatty acid, polyethylene glycol, talc, sodium stearyl fumarate, zinc stearate, castor oils, and waxes.

### Stabilizing Agents:

To prevent chemical interactions between amine groups and acidic polymers, amino acids can be included as stabilizers such as but not limited to glycine, arginine, etc.

### Colourants:

Various useful colourants include but are not limited to Food Yellow No. 5, Food Red No. 2, Food Blue No. 2, and the like, food lake colorants, and iron oxides.

### Flavoring Agents:

Flavoring agents, which can be used in this present invention, include but are not limited to materials having a natural, synthetic, or semisynthetic origin such as menthol, fruit flavors, citrus oils, peppermint oil, spearmint oil, and oil of wintergreen (methyl salicylate).

### Film-Forming Agents:

Various useful film-forming agents include but are not limited to: cellulose derivatives such as soluble alkyl- or hydroalkyl-cellulose derivatives such as methyl cellulose, hydroxymethyl cellulose, hydroxyethyl cellulose, hydroxypropyl cellulose, hydroxymethylethyl cellulose, hydroxypropyl methylcellulose (hypromellose or HPMC, different grades including HPMC 6 cps, HPMC 15 cps, HPMC 50 cps being available), sodium carboxymethylcellulose, etc., acidic cellulose derivatives such as cellulose acetate phthalate, cellulose acetate trimellitate and methylhydroxypropylcellulose phthalate, polyvinyl acetate phthalate, etc.; insoluble cellulose derivative such as ethyl cellulose and the like; dextrins; starches and starch derivatives; polymers based on carbohydrates and derivatives thereof; natural gums such as gum Arabic, xanthans, and alginates; polyacrylic acid; polyvinyl alcohol; polyvinyl acetate; polyvinylpyrrolidone; polymethacrylates and derivatives thereof (Eudragit™); chitosan and derivatives thereof; shellac and derivatives thereof; waxes; and fat substances.

If desired, films may contain additional adjuvants for the coating process such as plasticizers, polishing agents, colorants, pigments, antifoam agents, opacifiers, antisticking agents, and the like.

### Plasticizers:

Various useful plasticizers include but are not limited to castor oil, diacetylated monoglycerides, dibutyl sebacate, diethyl phthalate, glycerin, polyethylene glycol, propylene glycol, triacetin, and triethyl citrate. Also, mixtures of plasticizers may be utilized. The type of plasticizer depends upon the type of coating agent. A plasticizer is frequently present in an amount ranging from about 5% (w/w) to 30% (w/w) based on a total weight of the film coating.

An opacifier like titianium dioxide may also be present in an amount ranging from about 10% (w/w) to about 20% (w/w) based on the total weight of the coating. When coloured tablets are desired, the colour is normally applied in the coating. Consequently, colouring agents and pigments may be present in the film coating. Various colouring agents include but are not limited to iron oxides, which can be red, yellow, black or blends thereof.

Antiadhesives are frequently used in a film coating process to avoid sticking effects during film formation and drying. An example of a useful antiadhesive for this purpose is talc. The antiadhesive is present in the film coating in an amount of about 5% (w/w) to 15% (w/w) based upon the total weight of the coating.

Suitable polishing agents include polyethylene glycols of various molecular weights and mixtures thereof, talc, surfactants (e.g., glycerol monostearate and poloxamers), fatty alcohols (e.g., stearyl alcohol, cetyl alcohol, lauryl alcohol and myristyl alcohol) and waxes (e.g., carnauba wax, candelilla wax and white wax). In an embodiment, polyethylene glycols having molecular weights of 3,000-20,000 are employed.

In addition to the above coating ingredients, sometimes premixed coating material products, including those sold as OPADRY™ (supplied by Colorcon), for example Opadry blue 13B50579, will be used for convenience. These products require only mixing with a liquid before use.

### Solvents:

Various solvents that can be used in processes of preparation of pharmaceutical compositions of the present invention include but are not limited to water, methanol, ethanol, acidified ethanol, acetone, diacetone, polyols, polyethers, oils, esters, alkyl ketones, methylene chloride, isopropyl alcohol, butyl alcohol, methyl acetate, ethyl acetate, isopropyl acetate, castor oil, ethylene glycol monoethyl ether, diethylene glycol monobutyl ether, diethylene glycol monoethyl ether, dimethyl sulphoxide, dimethylformamide, tetrahydrofuran, and mixtures thereof.

### Preservatives:

Useful preservatives include alkyl esters of para-hydroxybenzoic acid like methylparaben and propylparaben, benzoates, hydantoin derivatives, propionate salts, sorbic acid, benzyl alcohol, imidazolidinyl urea, sodium dehydro acetate and a variety of quaternary ammonium compounds.

Cosolvents such as but not limited to glycerin, propylene glycol and stabilizers such as acids or acid derivatives example citric acid, sodium citrate may be employed for liquid oral dosage forms.

The compositions of the present invention may be prepared using any of mixing, comminution and tabletting techniques, many of which are well known in the art of pharmaceutical formulations.

In an embodiment the invention includes dosage forms, which are prepared using direct compression or dry granulation or wet granulation techniques.

The equipment suitable for processing the pharmaceutical compositions of the present invention include one or any combination of mechanical sifters, blenders, roller compactors, compression machines, rotating bowls or coating pans, etc.

### Processes for Preparing Pharmaceutical Compositions:

The present invention further relates to processes for manufacturing the pharmaceutical compositions of the present invention, wherein an embodiment of a process comprises:

1) Diluting active ingredient geometrically with diluent.

2) Sifting the geometric mixture through a sieve.

3) Adding polymer by geometric method to the mixture of 2).

4) Sifting a glidant through a sieve, adding to the mixture of 3), and blending.

5) Sifting a lubricant through a sieve and blending with the material of 4).

6) Compressing the final blend into a solid dosage form.

Tablets prepared as above can be subjected to an in vitro dissolution evaluation according to Test 711 “Dissolution” in United States Pharmacopoeia 29, to determine the rate at which the active substance is released from the dosage forms, and content of active substance can conveniently be determined in solutions using techniques such as high performance liquid chromatography.

The pharmaceutical dosage forms of the present invention are intended for oral administration to a patient in need thereof. In an embodiment, the compositions produce the memantine pharmacokinetic parameters: Cₘₐₓ values of about 10 ng/mL to about 20 ng/mL; AUC₀₋ₜ values of about 720 ng·hour/mL to about 1150 ng·hour/mL; and AUC_{0-α} values of about 850 ng·hour/mL to about 1330 ng·hour/mL; after oral administration of a single 10 mg memantine dose to healthy humans. These parameters are defined as follows:

Cₘₐₓ=maximum plasma concentration.

AUC₀₋ₜ=area under the plasma concentration versus time curve, from time zero to the last measurable concentration.

AUC_{0-∞}=area under the plasma concentrations versus time curve, from time zero to infinity.

In an embodiment the invention includes the use of packaging materials such as containers and lids of high-density polyethylene (HDPE), low-density polyethylene (LDPE) and or polypropylene and/or glass, and blisters or strips composed of aluminium or high-density polypropylene, polyvinyl chloride, polyvinylidene dichloride, etc.

Certain specific aspects and embodiments of the invention will be further described in the following examples, which are provided only for purposes of illustration and are not intended to limit the scope of the invention in any manner.

### EXAMPLE 1

### Memantine Hydrochloride 10 mg Tablets

| Ingredient | mg/Tablet |
|---|---|
| Memantine hydrochloride | 10 |
| Mannitol (Pearlitol ™ SD 200) | 75 |
| HPMC 50 cps | 160 |
| Colloidal silicon dioxide (Aerosil ™ | 1.75 |
| 200) | |
| Magnesium stearate | 3.25 |

Manufacturing Process:

1) Memantine hydrochloride was geometrically diluted with Pearlitol SD 200.

2) Sifted the geometric mixture through a #40 mesh sieve.

3) Hydroxypropyl methylcellulose (HPMC 50 cps) was added geometrically to the step 2) mixture.

4) Aerosil was added to the step 3) mixture and the total mixture was sifted through a #40 mesh sieve.

5) The mixture from step 4) was placed into a blender and blended for 10 minutes.

6) Magnesium stearate was sifted through a #80 mesh sieve, then was added to the blend of step 5) and blended for 5 minutes.

7) Final blend was compressed using 12×5 mm capsule shaped tooling.

The physicochemical properties of the active ingredient memantine hydrochloride used in the above example are given in Table 1.

TABLE 1

| Parameter | Value | |
|---|---|---|
| Bulk density | 0.317 | g/ml |
| Tapped density | 0.465 | g/ml |
| Hauser's ratio | 1.467 | |
| Compressibility index | 31.83 | |
| Particle size distribution: | | |
| D₍₁₀₎ | 10.744 | μm |
| D₍₅₀₎ | 60.545 | μm |
| D₍₉₀₎ | 210.482 | μm |

Above-prepared tablets were subjected to dissolution testing with the following dissolution conditions:

Volume of medium: 900 ml.

Apparatus: USP apparatus I, 100 rpm.

Medium: 0.1N HCl with NaCl solution (prepared by dissolving 12 g NaCl in 6 L of water and adjusting to pH 1.2 with 0.1 N HCl).

The results are given in Table 2.

TABLE 2

| | Cumulative % Drug Dissolved | | |
|---|---|---|---|
| | | pH 6.8 | |
| Time | | Phosphate | |
| (minutes) | 0.1N HCl | Buffer | HCl/NaCl Solution |
| 10 | 18 | 14 | 16 |
| 30 | 24 | 21 | 25 |
| 45 | 31 | 26 | 29 |
| 60 | 36 | 31 | 35 |
| 90 | 44 | 38 | 42 |

### EXAMPLE 2

### Memantine Hydrochloride 10 mg Tablets (Wet Granulation)

| Ingredient | mg/tablet |
|---|---|
| Memantine hydrochloride | 10 |
| Povidone K 30 (PVP K 30) | 10 |
| Mannitol | 105 |
| Isopropyl alcohol* | 15 |
| HPMC 50 cps | 115 |
| Talc | 5 |
| Colloidal silicon dioxide | 1.75 |
| Magnesium stearate | 3.25 |
| Opadry gray (03B17618)** | 7.5 |
| Isopropyl alcohol* | 45 |
| Methylene chloride* | 22.5 |

*Evaporates during processing.
**Opadry gray (03b17618) comprises HPMC 2910/hypromellose 6 cp, titanium dioxide, macrogol/peg 400, and iron oxide black and supplied by Colorcon.

Manufacturing Process:

(i) Memantine HCl is geometrically diluted with mannitol and sifted through the #40 mesh (ASTM).

(ii) To the above blend, PVPK30 is added and mixed properly. The blend is granulated using Isopropyl alcohol.

(iii) The granules are dried at 50° C. and then sifted through #30 mesh (ASTM). To this blend, HPMC 50 cps and colloidal silicon dioxide were added mixed thoroughly, and sifted through #30 mesh ASTM sieve.

(iv) The above blend is lubricated with magnesium stearate and compressed using 12×5 mm capsule shaped punches.

(v) The tablets thus obtained are coated using Opadry gray in a mixture of IPA and methylene chloride to produce a 3.0% w/w weight gain, using a pan-coater.

Above-prepared tablets are subjected to dissolution testing with the following conditions:

Medium: 0.1N HCl with NaCl solution (prepared by dissolving 12 g NaCl in 6 L of water and adjusting to pH 1.2 using 0.1 N HCl).

Volume of medium: 900 ml.

Apparatus: USP apparatus I, 100 rpm.

The results are given in Table 3.

TABLE 3

| Time (minutes) | Cumulative % Drug Dissolved |
|---|---|
| 10 | 20 |
| 30 | 23 |
| 45 | 31 |
| 60 | 38 |
| 90 | 50 |

### EXAMPLE 3

### Memantine Hydrochloride 10 mg Tablets

| Ingredient | mg/tablet |
|---|---|
| Memantine hydrochloride | 10 |
| Povidone K 30 (PVP K 30) | 7.5 |
| Mannitol | 147.5 |
| Isopropyl alcohol* | 24 |
| HPMC 50 cps | 75 |
| Talc | 5 |
| Colloidal silicon dioxide | 1.75 |
| Magnesium stearate | 3.25 |
| Opadry gray (03B17618) | 7.5 |
| Water* | 55 |

*Evaporates during processing.

Manufacturing process: same as that of Example 2.

Above-prepared tablets were subjected to dissolution testing with the following conditions:

Medium: 0.1N HCl with NaCl solution (prepared by dissolving 12 g NaCl in 6 L of water and adjusting to pH 1.2 using 0.1 N HCl).

Volume of medium: 900 ml.

Apparatus: USP apparatus I, 100 rpm.

The results are given in Table 4.

TABLE 4

| Time (minutes) | Cumulative % Drug Dissolved |
|---|---|
| 10 | 22 |
| 30 | 34 |
| 45 | 46 |
| 60 | 62 |
| 90 | 82 |

### EXAMPLE 4

### Memantine Hydrochloride 10 mg Tablets

| Ingredient | mg/Tablet |
|---|---|
| Memantine hydrochloride | 10 |
| Povidone K 30 (PVP K 30) | 7.5 |
| Mannitol | 147.5 |
| Isopropyl alcohol* | 70 |
| HPMC 50 cps | 75 |
| Talc | 5 |
| Colloidal silicon dioxide | 1.75 |
| Magnesium stearate | 3.25 |
| Opadry gray (03B17618) | 7.5 |
| Isopropyl alcohol* | 45 |
| Methylene chloride* | 22.5 |

*Evaporates during processing.

Manufacturing process: same as that of Example 2.

Above prepared tablets are subjected to dissolution testing with the following conditions:

Medium: 0.1N HCl with NaCl solution (prepared by dissolving 12 g NaCl in 6 L of water and adjusting to pH 1.2 using 0.1 N HCl).

Volume of media: 900 ml.

Apparatus: USP apparatus I, 100 rpm.

The results are shown in Table 5.

TABLE 5

| Time (minutes) | Cumulative % Drug Dissolved |
|---|---|
| 10 | 25 |
| 30 | 29 |
| 45 | 38 |
| 60 | 46 |
| 90 | 57 |

### EXAMPLE 5

### In Vivo Pharmacokinetic Study

The composition of Example 4 was compared with a commercial product, NAMENDA® 10 mg tablets, in an open label, balanced, randomized three treatment, three period, three sequence, analyst blinded, three-way crossover bioequivalence study with 10 day washout periods between each drug administration, under fasting conditions, using 18 healthy volunteers.

A summary of the determined pharmacokinetic parameters is given below:

| | | AUC₀₋ₜ | AUC_{0-∞} |
|---|---|---|---|
| Product | Cₘₐₓ (ng/ml) | (ng · hour/ml) | (ng · hour/ml) |
| NAMENDA ® | 15.83 | 910.12 | 1064.08 |
| Example 5 | 16.09 | 875.25 | 1041.45 |

### EXAMPLE 6

### Memantine Hydrochloride 10 mg Tablets

| Ingredient | mg/Tablet |
|---|---|
| Memantine hydrochloride | 10 |
| Lactose monohydrate | 206 |
| Corn starch | 8 |
| Povidone K 30 (PVP K 30) | 6 |
| Povidone K 30 (PVP K-30) | 2 |
| Isopropyl alcohol* | 55 |
| Talc | 6.8 |
| Corn starch | 8 |
| Colloidal silicon dioxide (Aerosil 200) | 1.2 |
| Magnesium stearate | 2 |
| Opadry gray (03B17618) | 8 |
| Isopropyl alcohol* | 37.17 |
| Methylene chloride* | 2.66 |
| Water* | 13.28 |

*Evaporates during processing.

Manufacturing process:

1) Memantine hydrochloride, lactose monohydrate, corn starch, and povidone (first quantity) were sifted through a 40 # mesh ASTM sieve.

2) Above sifted materials were mixed.

3) Povidione (second quantity) was dissolved in isopropyl alcohol to prepare binder solution.

4) Dry mix of step 2) was granulated using binder solution from step 3).

5) The granules were dried at 50° C. and then sifted through a #30 mesh ASTM sieve.

6) Corn starch (first quantity) and colloidal silicon dioxide were sifted through a #40 mesh ASTM sieve and talc and magnesium stearate were sifted through a #80 mesh ASTM sieve.

7) Step 6) materials, except magnesium stearate and talc, were added to the step 5) granules, blended and further lubricated by blending with talc and magnesium stearate.

8) Lubricated blend was compressed into tablets.

9) Opadry dispersion was prepared by dispersing Opadry into a mixture of solvents (water, isopropyl alcohol, and methylene chloride) and stirred for about 45 minutes.

10) The compressed tablets were coated with Opadry dispersion of step 9) to obtain a 3% weight gain.

Above-prepared tablets were subjected to dissolution testing with the following conditions:

Medium: 0.1N HCl with NaCl solution (prepared by dissolving 12 g NaCl in 6 L of water and adjusting to pH 1.2 using 0.1 N HCl).

Volume of medium: 900 ml.

Apparatus: USP apparatus I, 100 rpm.

The results are shown in Table 5.

TABLE 5

| Time (minutes) | Cumulative % Drug Dissolved |
|---|---|
| 5 | 64 |
| 10 | 96 |
| 15 | 98 |
| 30 | 99 |
| 45 | 99 |

### EXAMPLE 7

### Memantine Hydrochloride 10 mg Tablets

| Ingredient | mg/Tablet |
|---|---|
| Memantine hydrochloride | 10 |
| Povidone K 30 (PVP K 30) | 7.5 |
| Mannitol | 50.0 |
| Isopropyl alcohol* | 70 |
| Ethyl cellulose | 175.0 |
| Talc | 5 |
| Colloidal silicon dioxide | 1.75 |
| Magnesium stearate | 3.25 |
| Opadry gray (03B17618) | 7.5 |
| Isopropyl alcohol* | 45 |
| Methylene chloride* | 22.5 |

*Evaporates during processing.

Manufacturing process:

1) Mix memantine hydrochloride and mannitol in geometric proportion and sift through a #40 mesh ASTM sieve.

2) To the above blend, add PVP K30 and mix thoroughly.

3) Granulate the blend using isopropyl alcohol (first quantity).

4) Dry the granules at 50° C. and then sift through a #30 mesh ASTM sieve. To this blend add ethyl cellulose and colloidal silicon dioxide, mix thoroughly, and sift through a #30 mesh ASTM sieve.

5) Lubricate the above blend by blending with magnesium stearate and compress using 12×5 mm capsule shaped punches.

6) Coat the tablets obtained in step 5) using Opadry gray in a mixture of isopropyl alcohol (second quantity) and methylene chloride to produce a 3% w/w weight gain, using a pan-coater.

### EXAMPLE 8

### Memantine Hydrochloride 10 mg Tablets

| Ingredient | mg/Tablet |
|---|---|
| Memantine hydrochloride | 10 |
| Povidone K 30 (PVP K 30) | 7.5 |
| Mannitol | 100 |
| Isopropyl alcohol* | 70 |
| Ethyl cellulose | 75 |
| HPMC 50 cps | 75 |
| Talc | 5 |
| Colloidal silicon dioxide | 1.75 |
| Magnesium stearate | 3.25 |
| Opadry gray (03B17618) | 7.5 |
| Isopropyl alcohol* | 45 |
| Methylene chloride* | 22.5 |

*Evaporates during processing.

Manufacturing process: same as that of Example 7.

We claim:

1. A pharmaceutical composition comprising memantine or a salt thereof, wherein less than about 80 percent of contained memantine dissolves within about 60 minutes after immersion in a pH 1.2 aqueous medium.

2. The pharmaceutical composition of claim 1, wherein less than about 70 percent of contained memantine dissolves within about 60 minutes after immersion in a pH 1.2 aqueous medium.

3. The pharmaceutical composition of claim 1, wherein less than about 50 percent of contained memantine dissolves within about 60 minutes after immersion in a pH 1.2 aqueous medium.

4. The pharmaceutical composition of claim 1, producing memantine Cₘₐₓ values about 10 ng/mL to about 20 ng/mL, AUC₀₋ₜ values about 720 ng·hour/mL to about 1150 ng·hour/mL, and AUC_{0-α} values about 850 ng·hour/mL to about 1330 ng·hour/mL, after oral administration of a single 10 mg memantine or salt thereof dose to healthy humans.

5. The pharmaceutical composition of claim 1, further comprising at least one polymer.

6. The pharmaceutical composition of claim 1, further comprising a polymer comprising a cellulose derivative.

7. The pharmaceutical composition of claim 1, further comprising a polymer comprising hydroxypropyl methylcellulose.

8. The pharmaceutical composition of claim 1, wherein a salt of memantine comprises memantine hydrochloride.

9. The pharmaceutical composition of claim 8, wherein a ratio of D₁₀ to D₉₀ for memantine hydrochloride ranges from about 1:1 to about 1:30.

10. The pharmaceutical composition of claim 8, wherein a ratio of D₁₀ to D₉₀ for memantine hydrochloride ranges from about 1:15 to about 1:25.

11. The pharmaceutical composition of claim 8, wherein a ratio of D₁₀ to D₉₀ for memantine hydrochloride is about 20.

12. A process for preparing the composition of claim 1, the process comprising granulation using a nonaqueous solvent.

13. A pharmaceutical composition comprising memantine hydrochloride and a polymer, wherein less than about 80 percent of contained memantine dissolves within about 60 minutes after immersion in a pH 1.2 aqueous medium.

14. The pharmaceutical composition of claim 13, wherein a polymer comprises a cellulose derivative.

15. The pharmaceutical composition of claim 13, wherein a polymer comprises hydroxypropyl methylcellulose.

16. The pharmaceutical composition of claim 13, producing memantine Cₘₐₓ values about 10 ng/mL to about 20 ng/mL, AUC₀₋ₜ values about 720 ng·hour/mL to about 1150 ng·hour/mL, and AUC_{0-α} values about 850 ng·hour/mL to about 1330 ng·hour/mL, after oral administration of a single 10 mg memantine hydrochloride dose to healthy humans.

17. The pharmaceutical composition of claim 13, wherein a ratio of D₁₀ to D₉₀ for memantine hydrochloride ranges from about 1:1 to about 1:30.

18. The pharmaceutical composition of claim 13, wherein a ratio of D₁₀ to D₉₀ for memantine hydrochloride ranges from about 1:15 to about 1:25.

19. The pharmaceutical composition of claim 13, wherein a ratio of D₁₀ to D₉₀ for memantine hydrochloride is about 20.

20. A pharmaceutical composition comprising memantine hydrochloride and a cellulose derivative polymer, wherein less than about 80 percent of contained memantine dissolves within about 60 minutes after immersion in a pH 1.2 aqueous medium, the composition producing memantine Cₘₐₓ values about 10 ng/mL to about 20 ng/mL, AUC₀₋ₜ values about 720 ng·hour/mL to about 1150 ng·hour/mL, and AUC_{0-α} values about 850 ng·hour/mL to about 1330 ng·hour/mL, after oral administration of a single 10 mg memantine hydrochloride dose to healthy humans.

21. The pharmaceutical composition of claim 20, wherein a polymer comprises hydroxypropyl methylcellulose.
